(19) United States
(12) Patent Application Publication
(10) Pub. No.: US 2012/0130426 A1
(43) Pub. Date: May 24, 2012

(54) Orthosis to Correct Spinal Deformities

(76) Inventor: Matthew Thompson, Corte Madera, CA (US)

(21) Appl. No.: 13/282,381

(22) Filed: Oct. 26, 2011

Related U.S. Application Data
(63) Continuation of application No. 11/656,314, filed on Jan. 19, 2007, now abandoned.

(51) Int. Cl. A61B 17/70

(52) U.S. Cl. 606/248

(57) ABSTRACT

An orthosis for correcting spinal deformities by urging spinal vertebrae toward a vertical axis. The orthosis includes a series of retaining clamps fixed onto the spinous process of said vertebrae, each of said retaining clamps having guides for retaining at least one elastic rod.

### CROSS-RELATED APPLICATION

This application is a continuation of U.S. Ser. No. 11/656,314, filed Jan. 19, 2007, which is fully incorporated herein by reference.

### FIELD OF THE INVENTION

The present invention is directed to apparatus which is designed to be implanted within a patient exhibiting spinal disorders such as scoliosis in a way that requires less invasive surgery than prior devices of this kind and which do not involve fusion of the spinal column while achieving results which compare favorably to competitive apparatus.

### BACKGROUND

Scoliosis is a disease which deforms the spine affecting more girls than boys and manifesting itself during the teen years when significant growth is experienced. Scoliosis generally combines a horizontal torsion and flexion in a frontal plane and develops in three spatial dimensions. As noted, the disease generally begins with the growth phase as it is hypothesized that this is probably due to the rotation of one or two vertebral bodies.

Sufferers of scoliosis are generally treated initially with a rigid corset-like orthopedic brace. If this treatment proves unsuccessful, surgery is oftentimes resorted to. This involves the use of implantable apparatus including one and oftentimes two rods mounted on either side of the spinal column. If two rods are employed, anchoring means are provided positioning the rods in spaced-apart parallel alignment. Hooks or screws are employed to anchor the rods along the selected portion of the spinal column requiring intervention. Once installed, the anchors are rigidly locked to the associated rod to prevent relative motion there between and the entire arrangement supplemented with bone grafts causing fusion of the vertebrae in the area in which scoliosis has manifested itself.

Although spinal fusion can oftentimes largely correct a spinal deformity, such procedure is not without serious drawbacks. Spinal fusion can result in complications as the patient advances into adult life. Also, the surgery requiring the application of bone grafts and permanent fixation of supporting clamps to the transverse process is significantly invasive.

Others have suggested improvements to the orthosis described above. For example, U.S. Pat. No. 6,554,831 suggests a system that allows for intra-operative correction and micro-movement of the vertebrae despite implantation of a corrective rod. The '831 patent suggests use of a rigid rod that does not allow a patient to flex or extend post-operatively until the corrective rod is removed requiring additional surgery. Anchoring to the transverse process is also taught thus requiring significant invasive surgery and consequent fusion.

U.S. Pat. No. 5,672,175 suggests another approach which theoretically provides a patient with close to normal range of motion of the vertebrae by instrumenting the spine with elastic members pre-curved to correct the spinal deformity. Anchoring to the transverse process is also employed which, again, is a major drawback in performing the techniques suggested in the '175 patent. Further, this device theoretically overcomes the deformity with constant force applied by pre-curved correction members but this does not allow for resultant changes in the deformity or tissue relaxation. Because of the use of these pre-curve rods, the technique suggested in the '175 patent may actually result in a final deformity completely opposite to the original deformity due to tissue growth and relaxation.

U.S. Pat. No. 4,697,582 suggests a correction apparatus which employs an elastic rod or a pair of elastic rods exhibiting a memory shape of the corresponding part of a normal rachis, the rods being immobilized in rotation in each of its guidance openings. However, the mechanical assembly suggested in the '582 patent is appended to an area on each vertebrae between the spinal process and transverse process which again results in significant invasive surgery and results in fusion between vertebrae being established in the to be corrected region.

It is thus an object of the present invention to provide an appliance to correct spinal deformities while eliminating or significantly reducing the drawbacks of the prior art.

The present invention is further directed to an appliance to correct spinal deformities which does not result in spinal fusion and which significantly reduces the extent of invasive surgery and which substantially eliminates post operative drawbacks such as those exhibited by competitive apparatus.

These and further objects will be more readily apparent when considering the following disclosure and appended claims.

### SUMMARY OF THE INVENTION

The present invention is directed to an orthosis for correcting spinal deformities by urging spinal vertebrae towards a vertical axis, said orthosis comprising a series of retaining clamps fixed to the spinal (spinous) process of the vertebrae, each of the retaining clamps having guides for retaining at least one elastic rod. Ideally, the rod so implanted is immobilized in rotation within one of the guides. Two such rods can be employed to apply a corrective moment to rotational deformity of the spine in its axial plane.

### BRIEF DESCRIPTION OF THE DRAWING

FIG. 1 is a perspective view of a portion of a spine bearing the orthosis of the present invention.

FIG. 2 is a partial perspective view showing a portion of the spinous process of a vertebrae bearing a clamp for use in applying the orthosis of the present invention.

FIG. 3 is a perspective view, partially in cross section, showing a preferred embodiment of a correction rod useful in practicing the present invention.

FIG. 4 is a perspective view of a clamp bearing a correction rod illustrating one of the embodiments of the present invention.

FIG. 5 is a perspective view of clamping plates used as an alternative to the clamp shown in FIG. 4.

FIGS. 6 and 7 illustrate a healthy spine in the sagittal and anterior/posterior views, respectively, in conjunction with correction rods contemplated for use herein.

FIG. 8 is a perspective view of the clamping plates of FIG. 5 showing their application to the spinous process of a vertebrae bearing correction rods for use herein.

FIG. 9 is a side plan view of a series of vertebrae employing the clamping plate of FIG. 8.

FIG. 10 is a partial plan view of a correction rod for use herein showing one of the several ways to constrain motion of the rod once installed.

FIG. 11 is a perspective view of a corrective rod assembly proposed as a preferred embodiment of the present invention.

### DETAILED DESCRIPTION OF THE PREFERRED EMBODIMENTS

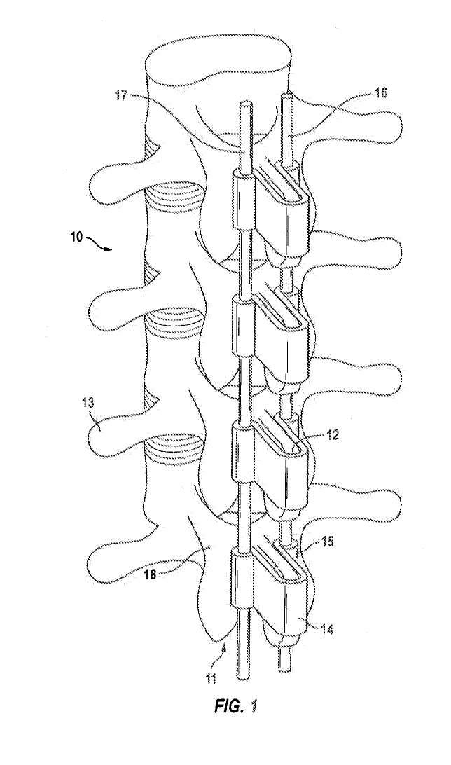

Referring first to FIG. 1, a portion of the human spine 10 is depicted having a series of vertebrae 11. Each vertebrae includes the spinous process 12 that transitions to the transverse process 13 through vertebral arch 18. It is important to note in carrying out the present invention that retaining clamps 14 are applied only to the spinous process thus significantly reducing the extent of invasive surgery and elimination or significantly reducing resultant spinal fusion. As noted previously, the prior art either applies retaining clamps to the transverse process 13 or at least to the vertebral arch 18 which for the reasons expressed herein, provides for spinal fusion, relatively significant invasive surgery and consequent patient discomfort and potentially long term physiological disadvantages.

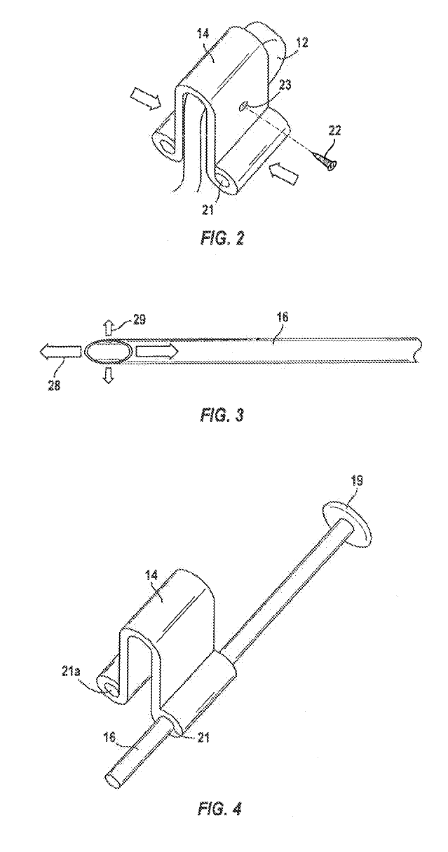

Retaining clamp 14 can be seen in greater detail by making reference to FIG. 2. Specifically, retaining clamp 14 is shown embracing spinous process 12. Retaining clamp 14 remains positioned thereon through the use of fixation screw 22 passing through opening 23 in retaining clamp 14 which thus passes within the body of the vertebrae at spinous process 12. A correction rod, the details of which will be discussed hereinafter, is intended to pass within and be carried by retaining clamp 14 at axial opening 21. When two correction rods are employed, a complimentary axial opening 21 a can be configured within clamp 14.

The present invention is intended to correct spinal deformities by generating corrective forces on any vertebrae in deviation from its anatomic or healthy position. This is done by providing corrective rods 16/17 within an axial openings 21/21a. In referring to FIG. 3, as a preferred embodiment, these elastic corrective rods can be designed to offer differing bending moments of inertia for each plane. This is important when dealing with a lateral deformity, such as scoliosis whereby rod 16 (FIG. 3) would generate lower forces on the vertebrae during flexion-extension of the spine but greater corrective forces on the lateral deformity. Such a feature would allow for easier or less painful natural motion while still providing sufficient force for reduction of the deformity. In referring to FIG. 3, rod 16 would thus be much stiffer and resistant to bending in the direction of arrow 28 than in the direction of arrow 29.

Again referring to rod 16, reference is made to FIGS. 6 and 7 showing rod 16 preconfigured in the shape of a healthy spine. Rod 16 is elastic or super elastic and can be made from a metal alloy, such as stainless steel, titanium or shaped memory alloy, or from a plastic such as PEEK. Rod 16 is fabricated to closely follow the contour of the spinous process of a healthy spine with no deformity. Any deformation from the original shape of the corrective rod 16 will result in a corrective force being applied. To generate a larger corrective force, these rods can be made larger or can be fabricated from a material with a higher modulus of elasticity or any combination thereof. Conversely, to generate a smaller corrective force, rod 16 can be made smaller or fabricated from a material with a lower modulus of elasticity or any combination thereof. Corrective rod 16 is also designed so that the spine will be free to move naturally without plastic deformation. As alternative embodiments, corrective rod 16 can generate forces sufficient to immediately reduce spinal deformity or can be sized to generate a force that is not sufficient to instantly reduce a deformity, but which will direct the spine back to a correct form over time, such as shown in FIGS. 6 and 7.

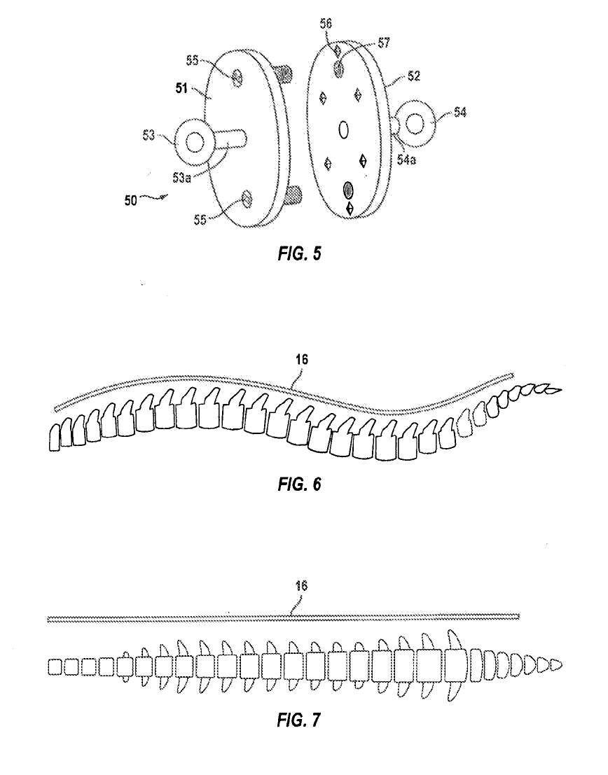

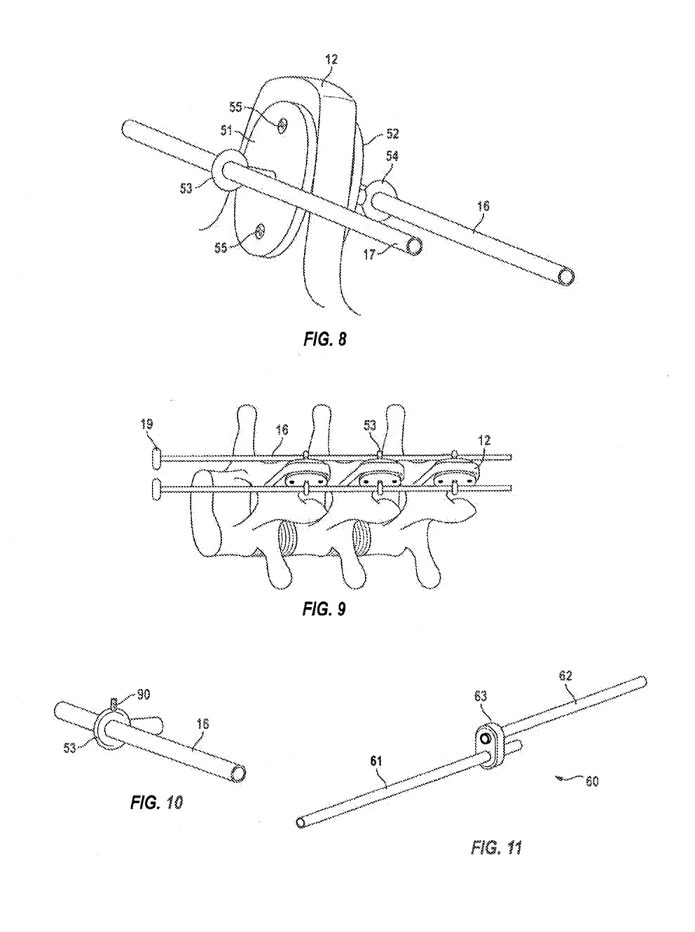

An alternative to the clamp of FIGS. 2 and 4 is shown in FIGS. 5 and 8. Specifically, clamp 50 is composed of two implantable clamping plates 51 and 52 which can be made from a metal alloy, such as stainless steel or titanium, or a plastic such as PEEK. These plates are secured onto the spinous process through the use of screws 55 which, together, act to generate a clamping force on the spinous process.

In installing clamp 50, a practitioner will position clamping plates 51 and 52 along the spinous process and generate pilot holes through tapped openings intended to receive threaded screws 55. Plates 51 and 52 are then screwed together and onto the spinous process and tightened to generate the appropriate fixation force enhanced by providing, as an optional expedient, spikes 56.

As was the case with clamp 14, plates 51 and 52 are provided with rod carriers 53 and 54 fabricated from a metal alloy such as titanium or stainless steel, or a plastic such as PEEK.

In a first embodiment, rod carriers 53 and 54 can be completely constrained to their respective clamping plates and thus not capable of rotational movement thereon. In a second embodiment, however, rod carriers 53 and 54 can be made free to rotate about their axial shafts 53a and 54a.

Rod carriers 53 and 54 are configured to constrain rods 16 and 17 either partially or completely and to promote the transfer of corrective forces exerted by these rods to the vertebrae through clamping plates 51 and 52 and spinous process. As noted, in a first embodiment, corrective rods 16 and 17 are partially constrained and free to translate axially through rod carriers 53 and 54. In a second embodiment, corrective rods 16 and 17 are completely restrained to rod carriers 53 and 54. An example of such constraint is shown in FIG. 10 whereby set screw 90 is shown passing through rod carrier 53 to constrain rod 16. Constraint can also be facilitated by other means such as by crimping rod carrier 53 onto rod 16 or, for that matter, by any other means which would be well appreciated by anyone skilled in this art. In this regard, as previously noted, rod 16 can be shaped to provide a reduced bending moment in one direction then another (FIG. 3) and movement constrain can be facilitated by sizing the opening within rod carriers 53 and 54 with regard to this non-circular cross section.

As noted previously, the present invention contemplates, as one of its embodiments, the ability of corrective rods 16 and 17 to freely translate axially through rod carriers 53 and 54 or in openings 21 and 21a. In doing so, however, a constraint must be placed on the unlimited motion of these rods so that they do not slide out from carriers or openings in the clamps and, in this regard, reference is made to FIG. 4. Specifically, end cap 19 can be provided at one or both terminal ends of rod 16 preventing rod 16 from inadvertently slipping from within opening 21. Again, an alternative constraint can be carried out by providing said screw 90 through a rod carrier, the latter completely constraining motion of rod 16 within the rod carrier (FIG. 10). When said screw 90 is employed, it is suggested that clamp 14 be selected as one close to the mid point of the instrumented region such that the relative displacement of the rod carriers on the corrective rod at extreme ends of the instrumented region is minimized during flexion and extension of the spine. As an alternative, one could select a vertebrae at the superior or inferior end of the instrumented region to carry out such constraint.

As a preferred embodiment, reference is made to FIG. 11. As background, it is recognized that as one moves his or her back into flexion, the spinous processes move apart. Ultimately this means that any correction rods should be longer than the original distance between the end points of the instrumentation constituting the present invention.

The embodiment of FIG. 11 is based upon the notion that in large-scale deformities with fixation along the spinous process, the difference in resting length and full-flexion is significant. One alternative, which cannot be adopted for obvious reasons, is to provide an extended length of correction rod extending way up and way down along a user's back. The more preferred alternative to which the FIG. 11 embodiment addresses is to “piggyback” corrective rods 61 and 62. Specifically, rod 62 is fixedly secured to fixture 63 by any common means such as by crimping or by set screw (not shown). However, rod 61 is free to slide in and out of fixture 63 noting that end caps will keep rod 61 from extending so far out of fixture 63 that engagement is lost. With this embodiment, one can distribute the excess length necessary for full flexion along the construct. Ideally, fixture 63 would be situated between clamps. It is further noted that fixture 63 could be replaced with a simple sheath that would fit about both rods 61 and 62 while performing the recited function.

In summary, the improvements in spinal deformity correction employing the present invention are manifest. Such correction, unlike the prior art, can be carried out with minimally invasive surgery while avoiding spinal fusion and the consequent physiological impairment resulting there from.

What is claimed is:

1. An orthosis for correcting spinal deformities by urging spinal vertebrae toward a vertical axis, said orthosis comprising a series of retaining clamps configured to be fixed only onto the spinous process of the vertebrae, each of said retaining clamps having a guide for retaining at least one elastic rod.

2. The orthosis of claim 1 wherein said at least one rod is immobilized in rotation within at least one guide.

3. The orthosis of claim 1 wherein the series of retaining clamps comprises a first retaining clamp configured to be fixed only onto a first spinous process of the vertebrae and a second retaining clamp configured to be fixed only onto a second spinous process of the vertebrae, further wherein the guide of the first retaining clamp is configured to retain a first elastic rod and the guide of the second retaining clamp is configured to retain a second elastic rod.

4. The orthosis of claim 1 wherein each guide is fixed and immovable with respect to said retaining clamps.

5. The orthosis of claim 1 wherein each guide is rotatable with respect to said retaining clamps.

6. The orthosis of claim 1 wherein each guide is configured to allow said elastic rod to remain slidable after implantation.

7. The orthosis of claim 1 wherein at least one retainer is provided for preventing removal of said rod from each guide.

8. The orthosis of claim 7 wherein said retainer comprises caps coupled to each end of said rod preventing said each end from passing through each guide.

9. The orthosis of claim 7 wherein said retainer comprises at least one set screw emanating from at least one retaining clamp for frictionally engaging said rod.

10. The orthosis of claim 3 further comprising a fixture for retaining the first elastic rod and the second elastic rod, wherein the fixture is configured to constrain the first elastic rod and further wherein the fixture is configured to allow the second elastic rod to remain slidable in the fixture after implantation in response to spinal flexion along said spinous process.

11. The orthosis of claim 10 wherein said fixture resides between adjacent retaining clamps.

12. An orthosis for correcting spinal deformities by urging spinal vertebrae toward a vertical axis, said orthosis comprising a series of retaining clamps configured to be fixed only onto the spinous process of the vertebrae, each of said retaining clamps comprising:
a first plate configured to be fixed on a first side of the spinous process of the vertebrae, the first plate having a first side and a second side, wherein the first plate is provided with least one post rotatably coupled to the first side of the first plate, wherein the post is provided with a guide for retaining an elastic rod;
a second plate configured to be fixed on a second side of the spinous process of the vertebrae, the second plate having a first side and a second side, wherein the second plate is provided with at least one post rotatably coupled to the first side of the second plate, wherein the post is provided with a guide for retaining an elastic rod; and
a plurality of engagement features configured to secure the first plate and the second plate onto the spinous process of the vertebrae.

13. The orthosis of claim 12 wherein the first plate and second plate are generally disc-shaped.

14. The orthosis of claim 12 wherein the plurality engagement features comprise a threaded screw set.

15. The orthosis of claim 12 wherein the second side of the first plate and the second side of the second plate are provided with a plurality of projections configured to engage the spinous process of the vertebrae.

16. The orthosis of claim 12 wherein the elastic rod is immobilized in rotation within the guide of each post of the first and second plate.

17. The orthosis of claim 12 wherein each guide is configured to allow said elastic rod to remain slidable after implantation.

18. The orthosis of claim 12 wherein at least one retainer is provided for preventing removal of the elastic rod from the guide of each post of the first and second plate.

19. The orthosis of claim 18 wherein said retainer comprises caps coupled to each end of the elastic rod preventing said each end from passing through the guide of each post of the first and second plate.

20. The orthosis of claim 1 wherein each of said retaining clamps comprises a first plate configured to be fixed on a first side of the spinous process of the vertebrae and a second plate configured to be fixed on a second side of the spinous process of the vertebrae.
